(19) United States
(12) Patent Application Publication
(10) Pub. No.: US 2021/0287236 A1
(43) Pub. Date: Sep. 16, 2021

(54) METHOD OF GAINING BIG DATA

(71) Applicant: DSM IP ASSETS B.V., Heerlen (NL)

(72) Inventors: Gerold BAECKER, Kaiseraugst (CH); Steffen GEIPEL, Kaiseraugst (CH)

(21) Appl. No.: 17/264,071

(22) PCT Filed: Jul. 16, 2019
(86) PCT No.: PCT/EP2019/069118

§ 371 Date: Jan. 28, 2021

(30) Foreign Application Priority Data
Jul. 31, 2018 (EP) 18186534.6

(51) Int. Cl. G06Q 30/02; G06Q 30/06; A61B 3/12; G06N 20/00

(52) U.S. Cl. CPC G06Q 30/0203; G06Q 30/0205; G06N 20/00; A61B 3/12; G06Q 30/0631

(57) ABSTRACT

The present invention relates to a method for gaining large amounts of data. The data is the output of a device which measures a predetermined parameter. Preferably, the predetermined parameter is the macular pigment optical density (MPOD). The gained data is preferably used to determine the effect of an intervention and/or treatment such as the oral intake of lutein and/or zeaxanthin.

### TECHNICAL FIELD

The present invention relates to the recruitment of participants for field studies and to the use of data gained in such studies. The invention also relates to the prevention and treatment of age-related macular degeneration (AMD).

### BACKGROUND OF THE INVENTION

In research, Big Data Analytics (BDA) has become a critical activity.

Algorithms suitable for the analysis of Big Data may be based on artificial intelligence (AI). Nowadays, such algorithms are easily accessible. However, commercially available AI based software can only be applied if suitable data is available.

The success of artificial intelligence (AI) depends on the quality of the available data. AI techniques can easily outperform traditional approaches, but only if sufficient quality data is available. Whereas the collection of data as such is relatively easy, it is a challenge to gain data which is meaningful and which has good quality.

One way of collecting data is doing a field study. When doing a field study, the recruitment of participants must be done very carefully. If criteria for exclusion/inclusion of participants are tailored to the purpose of the study, the data gained in a field study may be of good quality.

Several models are available for calculating the cost for recruiting participants. The total cost for each participant may range from $265 to $576 (Engstrom et al., Costs Associated With Recruitment and Interviewing of Study Participants in a Diverse Population of Community-Dwelling Older Adults, Nursing Research (2014), Volume 63, Issue 1, p. 63-67). Whereas such cost is acceptable for a relatively small study involving less than 500 participants, cost go beyond any reasonable limit when it comes to the collection of Big Data.

There is a need for a method for gaining a large amount of quality data in an easy and cost-effective manner. Ideally, the cost of recruiting one participant should be less than $100, preferably less than $10 or even less than $1. Quality means that the gained data is relevant for the goal/purpose of the study.

A goal/purpose of a study may be the prediction of cardiovascular risk factors. Popin et al. (Google Research, Mountain View, Calif., USA) trained deep-learning models using retinal fundus images from 48,101 patients from the UK Biobank and 236,234 patients from EyePACS. Whereas this seems to be a large number of patients, the authors of the Google study conclude “[ . . . ] the overall size of the dataset is relatively small for deep learning.” (Nature Biomedical Engineering, 158 Vol 2, March 2018, p. 161, right column, last paragraph).

Retinal fundus images can be used to investigate the eye's macula. Damage to the eye's macula has several potential causes. One factor is a lack of lutein. Lutein is primarily found in the macula of the eyes in humans and other animals. It is carried there by the blood and lymphatic systems following ingestion. The yellowish color of the macula is due to the ingestion of lutein. Humans are not capable of synthesizing lutein; it must be obtained from the diet. Lutein and zeaxanthin are postulated to function in a variety of ways. They may act as a short-wavelength (so-called blue) light filter, a signal transduction modulator, and an element in the structure of cell membranes. Accordingly, the lutein in the eye protects the eye from harmful effects of blue light. A lack of lutein increases the risk of macula damage such as age-related macular degeneration (AMD).

Other risk factors for AMD are ageing, family history, smoking, high cholesterol level, obesity and hypertension. Because the cause of AMD is multifactorial, there is no “one-size-fits-all” solution for preventing or treating AMD.

Age-related macular degeneration (AMD) can be prevented and—to a lesser degree—be treated by the oral intake of a dietary supplement comprising lutein (Dawczynski J, et al., Changes of macular pigment and drusen morphology in patients with lutein supplementation, Klin Monbl Augenheilkd. 2012; 229: 69-71). Another approach is to reduce exposure to blue light by wearing glasses with blue light filter lenses (Margrain T H et al., Do blue light filters confer protection against age-related macular degeneration? Prog Retin Eye Res. 2004 September; 23(5):523-31).

Whereas all of these solutions are helpful, there is a need for personalization.

Unfortunately, it is so far unknown which factors are decisive for providing tailor-made recommendations on how to treat or prevent AMD. One approach to unveil so far unknown correlations is to do a very large eye related field study and to analyze the thus gained Big Data with an AI based software.

Therefore, there is a need for gaining a large amount of eye-health data in an easy and cost-effective manner.

Various kinds of eye-health data are known. As AMD becomes more severe, more drusen occur and/or drusen become larger. Drusen are deposits which can be easily seen on an image taken by a fundus camera. Such camera allows to take a colored picture of the retina, optic disc and macula. Well-known providers of fundus camera are Zeiss® and EasyScan®.

MPS II® by the company Elektron Eye Technology® is a commercially available device for measuring macular pigment optical density (MPOD). MPOD values are measured on a scale from 0 to 1. The lower the MPOD value, the higher the risk of developing age-related macular degeneration (AMD). Measuring MPOD is a non-invasive and non-contact method.

Most probably, additional information could be extracted from MPOD data and/or fundus images using a suitable machine learning algorithm. A machine learning algorithm can only be applied if suitable Big Data is available. Thus, there is an urgent need for a tool to do a large or very large eye-health related field study in a cost-effective manner. Cost of recruiting one participant should be preferably less than $1.

### SUMMARY OF THE INVENTION

The problems underlying the present invention are solved by a method of gaining data and/or of doing a field study, said method comprising the steps:
a) selecting n stores,
b) providing at least n devices, wherein each of said devices is suitable for measuring at least one predetermined parameter,
c) providing each of the stores selected in step a) with at least one of the devices provided in step b), and
d) connecting each of the stores selected in step a) to a database such that the output of the devices which have been provided to said stores can be stored in said database,
   wherein n is an integer having a value of at least 100.

In the context of the present invention, “doing a field study” means carrying out a research project in the field (i.e. not in the lab, clinic etc.) in an empirical, systematic, controlled, analytical and objective (i.e. unbiased) manner. The term “gaining data” is broader and includes approaches which do not fully fulfill generally accepted scientific standards.

Preferably, the stores selected in step a) are stores belonging to the same market segment. Stores of the same market segment offer the same kind of products and/or services. Therefore, the clients entering said stores have the same kind of needs and often have a similar profile. By way of example, clients entering a Cuban cigar store are likely to be smokers, male, over 18 years old and prone to lung cancer. Such features are typical exclusion/inclusion criteria for participants of a field study.

In a field study, at least one relevant parameter is defined. The determined parameter(s) must be measurable. Depending on the technical nature of the parameter, a device can be used to measure the value of the parameter.

According to the invention, the stores selected in step a) are preferably equipped with the same kind of device. Same kind of device means that the devices are suitable for a measuring the same, predetermined parameter. Thus, referring to the above illustrative example and using blood pressure as predetermined parameter, all of the selected Cuban cigar stores are equipped with a sphygmomanometer.

Thus, the invention relates to a method of gaining data and/or of doing a field study, wherein the participants of said field study are selected among the clients visiting a store which is part of a network comprising multiple stores, and wherein at least one predetermined parameter is measured in said store. To do Big Data analytics (BDA), the data collected in the stores of the invention are stored in a data base, e.g. in a cloud database.

More specifically, the present invention relates to a network comprising n stores, wherein each of said n stores is equipped with at least one device which is suitable for a measuring at least one predetermined parameter, and wherein each of said n stores is connected to a database such that the output of said at least one device of each store can be stored in said database. In a preferred embodiment of the invention, said stores are optician stores, said at least one predetermined parameter is the macular pigment optical density and said at least one device is a device for measuring the macular pigment optical density.

A field study can be used to determine the effect of an intervention/treatment on the participants of the study. Therefore, the stores of the invention are preferably offering or selling at least one product which is suitable for effecting the value of the at least one predetermined parameter which can be measured by the devices provided to the stores of the invention. In the most preferred embodiment of the invention, the stores are optician stores, the at least one predetermined parameter is the macular pigment optical density, the at least one device is a device for measuring the macular pigment optical density and the stores are selling dietary supplements comprising lutein and/or zeaxanthin. The effect on the intake of lutein and/or zeaxanthin on the macular pigment optical density can then be observed at large scale and over a long period because clients tend to go back to the optician of their choice.

When applying the present invention, the cost of a field study is acceptable even if the study is very large. Cost effectiveness is achieved by easy recruitment of participants which fulfil certain inclusion criteria.

The measurement of the at least one predetermined parameter is preferably not a diagnostic method practised on the human or animal body. By way of example, the effect of cosmetic products could be investigated in a large field study by selecting stores owned by hairdressers and said stores being equipping with a device for measuring hair properties and said stores selling cosmetic products whose effects are to be determined.

### FIGURES

FIG. 1 shows the measurement of macular pigment optical density of the right eye of a 51-year old man. An MPOD value of 0.58 was measured using an MPS II® apparatus from the company Elektron Eye Technology®. MPS II uses the principle of heterochromatic flicker photometry (HFP). On the y-axis, frequency is shown in [Hz], whereas the green/blue ratio [dB] is indicated on the x-axis.

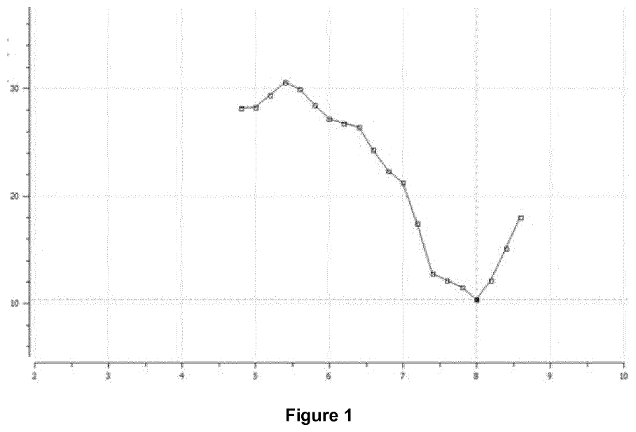

FIG. 2 shows an extract of a fundus image of the right eye of same 51-year old man (cf. FIG. 1).

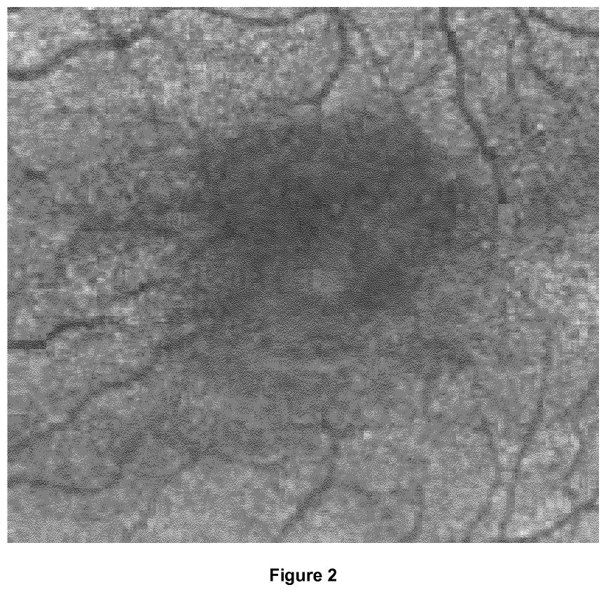

### DETAILED DESCRIPTION OF THE INVENTION

The problems underlying the present invention are solved by recruiting participants of a field study among clients visiting a store which is part of a network, said network comprising n stores.

In the context of the present invention, the term “store” is to be understood in a broad manner and includes stores that are selling services. However, the term “store” is preferably limited to physical shops which require the physical presence of the client. Virtual internet stores are preferably excluded. Health care facilities, hospitals, doctor's office and alike are also excluded. Preferably, drug stores and pharmacies are excluded, too.

A “retail store” is a store in which merchandise is sold primarily or exclusively to ultimate consumers. Stores and retail stores that belong to the same market segment are typically visited by a group of people who share one or more common characteristics. “Optician stores” are specialized in selling glasses and/or contact lenses to ultimate consumers.

A “network comprising n stores” comprises n stores which are directly or indirectly connected to the preferably same database, wherein n is an integer being a positive natural number. Preferably, the stores of such network belong to the same market segment and/or sell similar products or services.

When doing a field study, the parameter or the parameters to be measured are determined. Such parameter(s) is/are the basis of testable, and falsifiable scientific hypotheses. In this context, the term “predetermined parameter” is used. Such predetermined parameter may be, for example, blood pressure, macular pigment optical density, the color or thickness of hair etc.

Preferably, the stores of the network of the invention are selling at least one product which is suitable for effecting the value of said at least one predetermined parameter. Because many clients have the tendency to go back to the same stores, such a network of stores allows to determine the effect of an intervention/treatment on the participants of the study.

A device is then chosen which is preferably suitable for measuring the at least one predetermined parameter. Devices that are suitable for measuring the at least one predetermined parameter are defined by their functionality; they may or may not be identical. In a preferred embodiment, each store is provided with an identical device, e.g. with a MPS II® (available at Elektron Eye Technology®). Using identical devices make the gained scientific data more reliable and/or comparable.

In the context of the present invention, the term “device” refers preferably to a device for measuring the macular pigment optical density and/or to a fundus camera. When using a fundus camera, a camera employing Scanning Laser Ophthalmoscope (SLO) technology is preferably chosen. Such camera is commercially available at i-Optics® EasyScan®.

Each store of the network of the invention is equipped with at least one device which is suitable for a measuring the at least one predetermined parameter. In this context, the term “network” means that the at least one device of each store is connected to the database such that the output of the devices of all stores of the network can be transmitted to said database in order to be stored in said database. Preferably, the database is a cloud database.

The method and the network of the invention is particularly suitable for gaining a large amount of data. To gain Big Data, the network comprising n stores must be large. Therefore, n is preferably an integer having a value of at least 10, more preferably at least 500, even more preferably of at least 800 and most preferably of at least 1000.

The present invention also relates to the use of data gained by the method of the invention for providing a computer ontology, said computer ontology being stored in a database. The computer ontology of the invention is suitable for supervised or unsupervised machine learning and/or can be used as input for a machine learning algorithm, said algorithm being preferably a deep learning algorithm. Preferably, the data gained by the method of the invention is stored in non-volatile memory and/or is structured as ontology.

“Deep learning” is a specific machine learning method which is based on learning data representations, as opposed to task-specific algorithms. The term “machine learning algorithm” refers to a computer program that improves its performance when being trained. In the context of the present invention, supervised learning is preferred, although unsupervised learning is not excluded. Parameters of a neural network are initially set to random values. Then, for each entry, the prediction given by the algorithm is compared with the actual known value. Over time, parameters of the model are then modified to decrease the error rate.

### Particularly Preferred Embodiments of the Invention

A particularly preferred embodiment of the invention relates to a method of gaining data and/or of doing a field study, said method comprising the steps:
a) selecting n stores,
b) providing at least n devices, wherein each of said devices is suitable for measuring at least one predetermined parameter,
c) providing each of the stores selected in step a) with at least one of the devices provided in step b),
d) connecting each of the stores selected in step a) to a database such that the output of the devices which have been provided to said stores can be stored in said database,
e) measuring values of said at least one predetermined parameter in at least some of said n stores, and
f) storing the values measured in step e) in the database of step d)
   wherein n is an integer having a value of at least 100.

An also preferred embodiment of the invention relates to a network comprising n optician stores, wherein each of said n optician stores is equipped with at least one device for measuring the macular pigment optical density, and wherein each of said n optician stores is connected to a database such that the measured macular pigment optical densities can be stored in said database.

An also preferred embodiment of the invention relates to a network comprising n optician stores, wherein each of said n optician stores is equipped with at least one fundus camera, and wherein each of said n optician stores is connected to a database such that retinal images taken in said n optician stores can be stored in said database.

An even more preferred embodiment of the invention relates to a network comprising n optician stores that are selling at least one dietary supplement which comprises lutein and/or zeaxanthin, wherein each of said n optician store is equipped with at least one device for measuring the macular pigment optical density and/or a fundus camera, and wherein each of said n optician stores is connected to a database such that measured macular pigment optical densities can be stored in said database.

### EXAMPLES (HYPOTHETICAL)

### Example 1

1,000 sphygmomanometers are rented for a duration of 24 months to equip 1,000 cigar stores with a device for measuring blood pressure. Each of said devices is connected to a central database. During said 24 months, each store measures the blood pressure of 1000 of its clients (on a voluntary base, data anonymized). Said clients are (i) smokers, (ii) male, (iii) over 18 years old and do not need any travel reimbursement as they are in the store anyway. After 24 months, the field study is terminated, having collected the data of 1,000 stores*1,000 clients/store=1,000,000 clients.

### Comparative Example 2

A market research company is hired to recruit 1,000,000 participants for a study on respiratory health. Inclusion criteria are (i) smoker, (ii) male and (iii) over 18 years old. Said market research company charges a fee of 10$ per recruited participants. The participants are then invited to a research center for blood pressure measurement. The participants are reimbursed for travel expenses (average: 20$ per participants). Total cost: 1,000,000*10$*20$=200,000,000$.

### Example 3

A network of optician stores is established. Each store of said network is equipped with a device for measuring macular pigment optical density (MPOD). MPOD values of clients entering the stores are measured on a voluntary basis and free of charge. Furthermore, it is recorded whether the respective client is taking a dietary supplement that contains lutein and/or zeaxanthin. If he is taking such supplement, the amount and frequency is noted. Within 24 months, the client is revisiting the store at least once for adjustment of his glasses or to buy a contact lens cleaner. At this occasion, MPOD is measured for a second time (free of charge, on voluntary basis). In addition, the intake of the dietary supplement is noted/confirmed. The thus gained data is used as input for a machine learning algorithm.

1. Method of gaining data and/or of doing a field study, said method comprising the steps:
a) selecting n stores,
b) providing at least n devices, wherein each of said devices is suitable for measuring at least one predetermined parameter,
c) providing each of the stores selected in step a) with at least one of the devices provided in step b), and
d) connecting each of the stores selected in step a) to a database such that the output of the devices which have been provided to said stores can be stored in said database,
wherein n is an integer having a value of at least 100.

2. Method according to claim 1, wherein n is an integer having a value of at least 500, preferably of at least 800 and most preferably of at least 1000.

3. Method according to claim 1, wherein the stores selected in step a) are retail stores, and wherein said retail stores belong preferably to the same market segment.

4. Method according to claim 1, wherein each of the stores selected in step a) is selling at least one product which is suitable for effecting the value of said at least one predetermined parameter which can be measured by the devices provided to said stores in step c).

5. Method according to claim 1, wherein the measurement of said at least one predetermined parameter is not a diagnostic method practised on the human or animal body.

6. Network comprising n stores, wherein each of said n stores is equipped with at least one device which is suitable for measuring at least one predetermined parameter, and wherein each of said n stores is connected to a database such that the output of said at least one device of each store can be stored in said database.

7. Network according to claim 6, wherein n is an integer having a value of at least 10, more preferably at least 500, even more preferably of at least 800 and most preferably of at least 1000.

8. Network according to claim 6, wherein said stores are retail stores and/or wherein said stores belong to the same market segment, and wherein said stores are preferably optician stores selling glasses and/or contact lenses.

9. Network according to claim 6, wherein said at least one predetermined parameter is a parameter of the eye and/or wherein said at least one predetermined parameter is the macular pigment optical density.

10. Network according to claim 6, wherein each of said stores is equipped with at least one device for measuring the macular pigment optical density and/or wherein each of said stores is equipped with at least one fundus camera.

11. Network according to claim 6, wherein each of said stores is selling and/or offering at least one product which is suitable for increasing the macular pigment optical density, and wherein said at least one product is preferably a dietary supplement which comprises preferably lutein and/or zeaxanthin.

12. Use of data gained by the method according to claim 1 for providing a computer ontology, said computer ontology being stored in a database.

13. Use according to claim 12, wherein said computer ontology is stored in non-volatile memory.

14. Use according to claim 12, wherein said computer ontology is suitable for supervised or unsupervised machine learning and/or can be used as input for a machine learning algorithm, said algorithm being preferably a deep learning algorithm.

15. Method, network or use according to claim 1, wherein said database is a cloud database.
